(19) United States
(12) Patent Application Publication
(10) Pub. No.: US 2021/0008361 A1
(43) Pub. Date: Jan. 14, 2021

(54) MEDICAL TUBING SYSTEM

(71) Applicant: Marc D. ARONSON, Middlefield, CT (US)

(72) Inventor: Marc D. ARONSON, Middlefield, CT (US)

(21) Appl. No.: 16/507,939

(22) Filed: Jul. 10, 2019

(51) Int. Cl. A61M 39/10; A61M 16/08

(52) U.S. Cl. CPC A61M 39/10; A61M 2039/1061; A61M 16/0875

(57) ABSTRACT

A medical tubing system whereby the tubing is configured to break apart when subjected to forces, thereby reducing the risk of patient strangulation through accidental entanglement or deliberate self-harm attempt.

### BACKGROUND OF THE INVENTION

Medical tubing is used in many different applications in the healthcare field. In general terms, it is used to convey either liquids or gases between a container and a patient. Medical tubing is made from medical grade plastics, for example, polyvinyl chloride, polyethylene, polypropylene, nylon, or other similar materials, consistent with Federal Drug Administration (FDA) standards. It does not react with the liquids or gases flowing through it. Existing medical tubing is configured as a continuous length with attachment means at either end. In most cases, the tubing interacts with the patient via a hollow needle (when inserted into a patient's vein, this is known as an intravenous application), or via a catheter, or via a cannula. It interacts with the container via a specialized connector, which may be configured to allow the movement of fluids or gases in a single direction only. Examples of liquids conveyed by medical tubing include blood products and medications, delivered intravenously, and urine, removed from a patient through catheterization. Oxygen delivered through a nasal cannula is an example of a gas conveyed through medical tubing.

While medical tubing provides great benefits in the delivery of healthcare services, it can also present dangers to certain patients. Because of the startling high incidence of suicides by hospitalized patients, in 2016 the Joint Commission on Accreditation of Healthcare Organizations (JCAHO) issued a Sentinel Event Alert requiring hospitals to have a suicide risk detection and treatment strategy for all patients in all hospital settings. Because the method of suicide is often by hanging, in 2017 the Center for Medicare and Medicaid Services (CMS) enhanced the JCAHO standard, requiring hospitals to minimize ligature risks in areas specifically designed to treat behavioral health patients. To date, hospitals have been able to modify fixtures such as hinges, door edges, and exposed piping to eliminate attachment points for rope, cords, belts, or other devices that could be made into a noose. However, no one has adequately addressed the primary device used as a noose (due to ubiquity in healthcare settings): medical tubing.

In many settings, behavioral health patients deemed to be at risk who require the use of medical tubing are placed on one-to-one watch. While direct observation does reduce the risk of a patient using medical tubing to self-strangulate, it is not fool-proof. In addition, patients may not be properly identified as being at risk, and so one-to-one observation may not be in place. And even when one-to-one observation is used, it is an expensive and inefficient means for preventing suicide attempts.

In addition to self-harm, patients may also be accidentally harmed by medical tubing, by becoming inadvertently entangled. Because accidental entanglement is not limited to behavioral health patients, one-to-one observation is not a solution.

It thus is shown that there is a need for an improved medical tubing system that can be used to minimize, if not completely eliminate, the risk of harm to a patient from strangulation, whether intentional or accidental.

### SUMMARY OF THE INVENTION

The present invention is an improved medical tubing system engineered to break apart into relatively short lengths upon being subjected to minimal pulling or stretching forces. The resulting lengths are not long enough to form a noose, nor to present an accidental entanglement risk.

The medical tubing of the present invention in made of the same materials as conventional medical tubing. It uses the same attachment means to interact with the patient and the container. As with conventional medical tubing, it is leak-proof under gravity feed, and can withstand internal pressures of gases used in medical applications.

The medical tubing of the present invention exhibits its breakaway characteristic in one of two ways: by modifying a continuous length of tubing, or by using specialized connectors to create a longer length of tubing from multiple shorter lengths. In the first configuration, the modifications provide break points formed into the tubing, whereby the tubing may more easily separate at each such point. In the second configuration, the specialized connectors, which permit fluids or gases to pass therethrough, are configured to release from the tubing, or to themselves come apart.

Other features and advantages of the invention are described below.

### DESCRIPTION OF THE DRAWINGS

The two variants of FIG. 1 (FIGS. 1A and 1B) are plan side views of one embodiment of the medical tubing of the present invention. In both variants, the medical tubing is manufactured in a continuous length, with portions of the tubing wall being thinner relative to the remaining tubing wall located at periodic positions along the length of the tubing. Separation of segments of the tubing occurs by rupturing the tubing at a location of thinner tubing wall.

FIG. 1A depicts the medical tubing whereby the periodic thinner portions are created during the formation of the tubing.

FIG. 1B depicts the medical tubing whereby the periodic thinner portions are created mechanically after the tubing has been formed.

The five variants of FIG. 2 (FIGS. 2A, 2B, 2C, 2D, and 2E) are plan side views of another embodiment of the medical tubing of the present invention. In all five variants, the medical tubing is comprised of a plurality of relatively short segments, with each pair of segments joined by a single connector element. Separation of segments of the tubing occurs by an end of a connector element becoming disengaged from a tubing segment.

FIG. 2A depicts the connector element configured as a male-male connector, whereby each end of the connector is fitted into an end of a tube segment.

FIG. 2B depicts the connector element configured as a male-male connector with an enlarged central portion to control depth of insertion, whereby each end of the connector is fitted into an end of a tube segment.

FIG. 2C depicts the connector element configured as a female-female connector, whereby each end of the connector is fitted over an end of a tube segment.

FIG. 2D depicts the connector element configured as a female-female connector with a narrowed central portion to control depth of insertion, whereby each end of the connector is fitted over an end of a tube segment.

FIG. 2E depicts the connector element configured as a male-female connector, whereby the male end of the connector is fitted into an end of one tube segment and the female end of the connector is fitted over an end of the other tube segment.

The six variants of FIG. 3 (FIGS. 3A, 3B, 3C, 3D, 3E, and 3F) are plan side views of yet another embodiment of the medical tubing of the present invention. In all six variants, the medical tubing is comprised of a plurality of relatively short segments, with each pair of segments joined by a multi-part connector element. Separation of segments of the tubing occurs by one of the connector element components becoming disengaged from another of the connector element components.

FIG. 3A depicts the connector element configured as a male-female-male multi-part connector, whereby each of the male connector components is fitted at one end into an end of a tube segment, and fitted at the other end into the female connector component.

FIG. 3B depicts the connector element configured as a male-female-male multi-part connector, whereby each of the male connector components is fitted at one end into an end of a tube segment, and fitted at the other end into the female connector component; moreover, each male connector component has an enlarged central portion to control depth of insertion.

FIG. 3C depicts the connector element configured as a female-male-female multi-part connector, whereby each of the female connector components is fitted at one end over an end of a tube segment, and fitted at the other end over an end of the male connector component.

FIG. 3D depicts the connector element configured as a female-male-female multi-part connector, whereby each of the female connector components is fitted at one end over an end of a tube segment, and fitted at the other end over an end of the male connector component; moreover, the male connector component has an enlarged central portion to control depth of insertion.

FIG. 3E depicts the connector element configured as a female-male multi-part connector, whereby one end of the female connector component is fitted over an end of a tube segment, the other end of the female connector component is fitted over one end of the male connector component, and the other end of the male connector component is fitted into an end of another tube segment.

FIG. 3F depicts the connector element configured as a pair of female-male multi-part connectors, whereby the female end of one of the female-male connector components is fitted over an end of a tube segment, the male end of the female-male connector component is fitted into the female end of the other female-male connector component, and the male end of the other female-male connector component is fitted into an end of another tube segment.

### DETAILED DESCRIPTION OF INVENTION

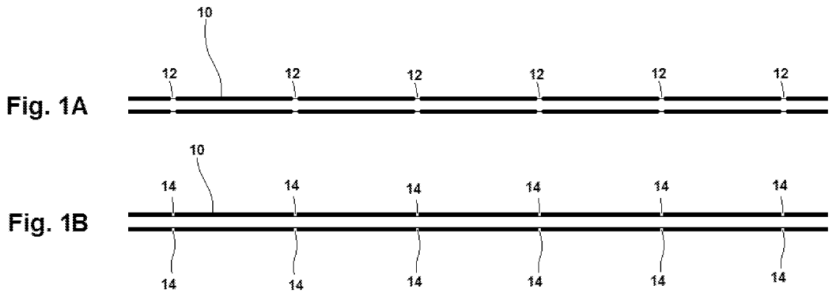

In one embodiment of the present invention, the medical tube 10 is manufactured in a continuous length, with periodic alterations in the manufacturing process resulting in a thinner tubing wall 12 at designated locations along the tubing 10 relative to the remaining tubing 10. See FIG. 1A. The thinner wall portion 12 of the tubing 10 is rupturable when subjected to a pre-determined pulling or stretching force, said force being less than the force needed to rupture the remaining portions of the tubing 10. The thinning of the tubing wall 10 could be achieved by using a variable diameter extrusion mold, or by injecting bursts of air into the extrusion process, or by any other means known in the art. The result is a length of tubing 10 that requires no further processing in order to be used.

In another embodiment, the medical tubing 10 is manufactured in a continuous length, as is the case with conventional medical tubing. However, the tubing 10 is then further processed to introduce break points 14 along its length. See FIG. 1B. This may be accomplished by mechanical scoring, or by using lasers, or a heat source, or by any other means known in the art. As described above, the break points 14 formed into the tubing 10 are rupturable when subjected to a pre-determined pulling or stretching force.

When releasable connectors are used, the medical tubing 10 of the present invention is first manufactured in the traditional manner, and then cut into desired lengths. These lengths typically may be between one inch and fifteen inches long. The lengths of tubing 10 are then joined together by using connectors, which are friction fit to the lengths of tubing 10 or joined thereto by an adhesive having weak attachment properties. The connectors are configured to separate from the lengths of tubing 10 when subjected to pre-defined forces, said forces being less than the force needed to rupture the tubing 10.

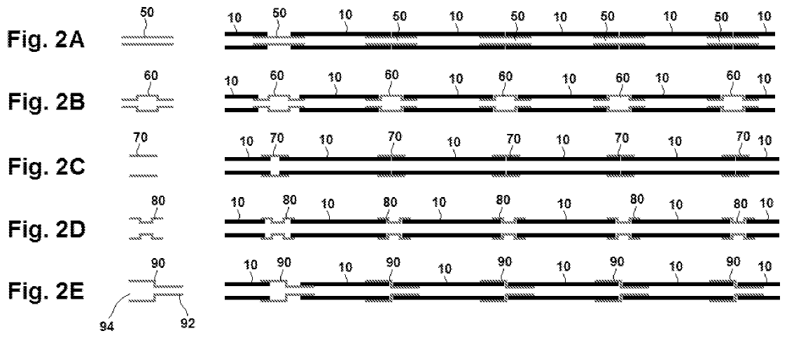

In one embodiment, the connector is a male-male connector 50, whereby each end of the connector 50 is inserted into the open end of a length of tubing 10. See FIG. 2A (FIG. 2A shows the connector 50 apart from the tubing 10, partially inserted into the tubing 10, and then fully inserted into the tubing 10). A variant of this embodiment uses a male connector with an enlarged central portion 60, to control depth of insertion. See FIG. 2B (FIG. 2B shows the connector 60 apart from the tubing 10, partially inserted into the tubing 10, and then fully inserted into the tubing 10). In another variant of this embodiment, the connector is a female-female connector 70, whereby each end of the connector 70 is placed over the open end of a length of tubing 10. See FIG. 2C (FIG. 2C shows the connector 70 apart from the tubing 10, partially inserted over the tubing 10, and then fully engaged with the tubing 10). Yet another variant of this embodiment uses a female connector with a restricted central portion 80, to control depth of insertion. See FIG. 2D (FIG. 2D shows the connector 80 apart from the tubing 10, partially inserted over the tubing 10, and then fully engaged with the tubing 10). In yet another variant of this embodiment, the connector is a female-male connector 90, whereby the male end 92 of the connector 90 is placed into the open end of one length of tubing 10, and the female end 94 of the connector 90 is placed over the open end of a second length of tubing 10. See FIG. 2E (FIG. 2E shows the connector 90 apart from the tubing 10, partially engaged with the tubing 10, and then fully engaged with the tubing 10).

When separable connectors are used, the medical tubing 10 of the present invention is first manufactured in the traditional manner, and then cut into desired lengths, as described above. The separable connectors are then friction fit to the lengths of tubing 10 or joined thereto by an adhesive having strong attachment properties. The separable connectors are configured to remain attached to the lengths of tubing 10 but to themselves come apart when subjected to pre-defined separation forces, said forces being less than the force needed to rupture the tubing 10. Each separable connector component is friction fit or snap-fit to its adjoining connector component, or joined to its adjoining connector component by an adhesive having weak attachment properties. The friction fit or adhesive attaching the separable connectors to the lengths of tubing 10 are sufficient to withstand the separation forces, while the friction fit, snap-fit, or adhesive attaching the connector components to each other are insufficient to withstand the separation forces.

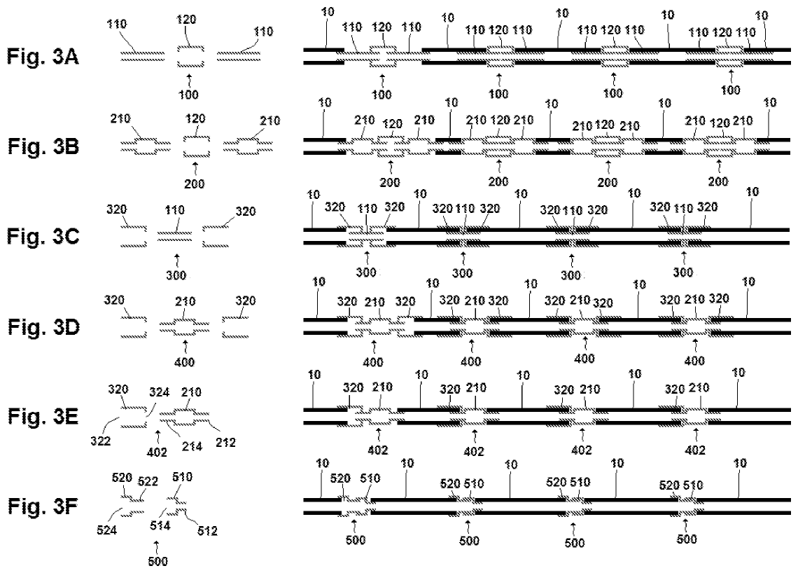

In one embodiment of the separable connector configuration, the separable connector is a male-female-male connector 100, whereby one end of each male connector component 110 is inserted into the open end of a length of tubing 10, and the other ends of the male connector components 110 are inserted into the female connector component 120 interposed between the male connector components. See FIG. 3A (FIG. 3A shows the connector 100 with its components 110,120,110 separated from the tubing 10 and from each other, then with the components 110,120,110 partially assembled and partially engaged with the tubing 10, and then with the components 110,120,110 fully assembled and fully engaged with the tubing 10). A variant of this embodiment uses male connector components 210 with enlarged central portions, to control depth of insertion. See FIG. 3B (FIG. 3B shows the connector 200 with its components 210,120,210 separated from the tubing 10 and from each other, then with the components 210,120,210 partially assembled and partially engaged with the tubing 10, and then with the components 210,120,210 fully assembled and fully engaged with the tubing 10).

In another variant of this embodiment, the connector is a female-male-female connector 300, whereby one end of each female connector component 320 is placed over the open end of a length of tubing 10, and the ends of the male connector component 110, which is interposed between the female connector components, are inserted into the other ends of the female connector components 320, joining them together. See FIG. 3C (FIG. 3C shows the connector 300 with its components 320,110,320 separated from the tubing 10 and from each other, then with the components 320,110,320 partially assembled and partially engaged with the tubing 10, and then with the components 320,110,320 fully assembled and fully engaged with the tubing 10). Another variant of this embodiment uses a male connector component 210 with an enlarged central portion, to control depth of insertion. See FIG. 3D (FIG. 3D shows the connector 400 with its components 320,210,320 separated from the tubing 10 and from each other, then with the components 320, 210,320 partially assembled and partially engaged with the tubing 10, and then with the components 320,210,320 fully assembled and fully engaged with the tubing 10).

In yet another variant of this embodiment, the connector is a female-male connector 402, whereby the first end 322 of the female connector component 320 is placed over the open end of one length of tubing 10, the first end 212 of the male connector component 210 is inserted into the open end of a second length of tubing 10, and the second end 214 of the male connector component 210 is inserted into the second end 324 of the female connector component 320, joining them together. See FIG. 3E (FIG. 3E shows the connector 402 with its components 320,210 separated from the tubing 10 and from each other, then with the components 320,210 partially assembled and partially engaged with the tubing 10, and then with the components 320,210 fully assembled and fully engaged with the tubing 10).

In yet another variant of this embodiment, the connector is a double female-male connector 500, whereby each connector component has a female end and a male end. The female end 524 of the first connector component 520 is placed over the open end of one length of tubing 10; the male end 512 of the second connector component 510 is inserted into the open end of a second length of tubing 10; and the male end 522 of the first connector component 520 is inserted into the female end 514 of the second connector component 510, joining them together. See FIG. 3F (FIG. 3F shows the connector 500 with its components 520,510 separated from the tubing 10 and from each other, then with the components 520,510 partially assembled and partially engaged with the tubing 10, and then with the components 520,510 fully assembled and fully engaged with the tubing 10).

What has been described and illustrated herein are preferred embodiments of the medical tubing system of the present invention along with some it its variations. The terms, descriptions and figures used herein are set forth by way of illustration only and are not meant as limitations. Those skilled in the art will recognize that many variations are possible within the spirit and scope of the invention in which all terms are meant in their broadest, reasonable sense unless otherwise indicated. Other embodiments not specifically set forth herein are also contemplated.

1. (canceled)

2. (canceled)

3. (canceled)

4. (canceled)

5. A medical tubing system comprising
three or more tubing segments, each said tubing segment having a length, a first opening located at a first end, a second opening located at a second end, a wall, an outside diameter, and an inside diameter, with all of the tubing segments forming a single length of tubing when joined together; and
a plurality of connectors, each said connector configured to join a pair of tubing segments together;
wherein the length of each said tubing segment is between one inch and fifteen inches, and
each of the plurality of connectors joins its corresponding pair of tubing segments together by being interposed therebetween and attached thereto;
whereby when said length of tubing is subjected to a breaking force, said breaking force being insufficient to rupture any single tubing segment, at least one pair of tubing segments will become separated from each other at a location of a connector joining said pair of tubing segments.

6. The medical tubing system of claim 5 wherein
when the length of tubing is subjected to the breaking force, at least one of the plurality of connectors will separate from at least one of the pair of tubing segments to which said connector had been attached.

7. The medical tubing system of claim 5 wherein
each of the plurality of connectors is retained in connection with the pair of tubing segments to which it is attached by frictional forces, said frictional forces being less than the breaking force.

8. The medical tubing system of claim 5 wherein
each of the plurality of connectors is retained in connection with the pair of tubing segment to which it is attached by an adhesive, said adhesive being unable to withstand the breaking force.

9. The medical tubing system of claim 5 wherein
at least one of the plurality of connectors is a male-male connector, having a first end and a second end, with the first end of said male-male connector configured to be inserted into one of the openings of one of the pair of tubing segments to which said connector is attached and the second end of said male-male connector configured to be inserted into one of the openings of the other of said pair of tubing segments.

10. The medical tubing system of claim 5 wherein
at least one of the plurality of connectors is a female-female connector, having a first end and a second end, with the first end of said female-female connector configured to be inserted over one of the ends of one of the pair of tubing segments to which said connector is attached and the second end of said female-female connector configured to be inserted over one of the ends of the other of said pair of tubing segments.

11. The medical tubing system of claim 5 wherein
at least one of the plurality of connectors is a female-male connector, having a first end and a second end, with the first end of said female-male connector configured to be inserted over one of the ends of one of the pair of tubing segments to which said connector is attached and the second end of said female-male connector configured to be inserted into one of the ends of the other of said pair of tubing segments.

12. The medical tubing system of claim 5 wherein
each of the plurality of connectors is comprised of two or more connector components joined together.

13. The medical tubing system of claim 12 wherein
when the length of tubing is subjected to the breaking force, at least two of the connector components of at least one connector will separate from each other.

14. The medical tubing system of claim 12 wherein
for each of the plurality of connectors, each connector component is retained in connection with the connector component to which it is joined by frictional forces, said frictional forces being less than the breaking force.

15. The medical tubing system of claim 12 wherein
for each of the plurality of connectors, each connector component is retained in connection with the connector component to which it is joined by an adhesive, said adhesive being unable to withstand the breaking force.

16. The medical tubing system of claim 12 wherein
for each of the plurality of connectors, each connector component is retained in connection with the connector component to which it is joined by a snap-fit mechanism, said snap-fit mechanism being unable to withstand the breaking force.

17. The medical tubing system of claim 12 wherein
for each of the plurality of connectors, at least one connector is a male-female-male connector, having a pair of male connector components and a female connector component, with each said connector component having a first end and a second end;
wherein the female connector component is positioned between the two male connector components, the first end of one of the male connector components is inserted into an open end of one of the pair of tubing segments with which said connector is associated, the first end of the other of the male connector components is inserted into an open end of the other of said pair of tubing segments, the second end of one of the male connector components is inserted into the first end of the female connector component, and the second end of the other of the male connector components is inserted into the second end of the female connector component;
whereby when the tubing is subjected to the breaking force, said female connector component will separate from at least one of said male connector components.

18. The medical tubing system of claim 12 wherein
at least one connector is a female-male-female connector, having a pair of female connector components and a male connector component, with each said connector component having a first end and a second end;
wherein the male connector component is positioned between the two female connector components, the first end of one of the female connector component is placed over an end of one of the pair of tubing segments with which said connector is associated, the first end of the other of the female connector components is placed over an end of the other of said pair of tubing segments, the first end of the male connector component is inserted into the second end of one of said female connector components, and the second end of the male connector component is inserted into the second end of the other said female connector component;
whereby when the length of tubing is subjected to the breaking force, said male connector component will separate from at least one of said female connector components.

19. The medical tubing system of claim 12 wherein
at least one connector is a female-male connector, having a female-female connector component and a male-male connector component, with each said connector component having a first end and a second end;
wherein the first end of said female-female connector component is placed over an end of one of the pair of tubing segments with which said connector is associated, the first end of the male-male connector component is inserted into an opening of the other of said pair of tubing segments, and the second end of the male-male connector component is inserted into the second end of the female-female connector component;
whereby when the length of tubing is subjected to the breaking force, said male-male connector component will separate from said female-female connector component.

20. The medical tubing system of claim 12 wherein
at least one connector is a double female-male connector, having a first female-male connector component and a second female-male connector component, with each said connector component having a first end and a second end;
wherein the first end of said first female-male connector component is placed over an end of one of the pair of tubing segments with which said connector is associated, the second end of said second female-male connector component is inserted into an opening of the other of said pair of tubing segments, and the second end of the first female-male connector component is inserted into the first end of the second female-male connector component;
whereby when the length of tubing is subjected to the breaking force, said first female-male connector component will separate from said second female-male connector component.

21. The medical tubing system of claim 12 wherein
at least one connector is a female-female connector, having a female-male connector component and a female-female connector component, with each said connector component having a first end and a second end;
wherein the first end of said female-male connector component is placed over an end of one of the pair of tubing segments with which said connector is associated, the second end of said female-female connector component is placed over an end of the other of said pair of tubing segments, and the second end of the female-male connector component is inserted into the first end of the female-female connector component;
whereby when the length of tubing is subjected to the breaking force, said female-male connector component will separate from said female-female connector component.

22. The medical tubing system of claim 12 wherein
at least one connector is a male-male connector, having a male-male connector component and a female-male connector component, with each said connector component having a first end and a second end;
wherein the first end of said male-male connector component is inserted into an opening in an end of one of the pair of tubing segments with which said connector is associated, the second end of said female-male connector component is inserted into an opening in an end of the other of said pair of tubing segments, and the second end of the male-male connector component is inserted into the first end of the female-male connector component;
whereby when the length of tubing is subjected to the breaking force, said male-male connector component will separate from said female-male connector component.
